(19) United States
(12) Patent Application Publication
(10) Pub. No.: US 2014/0073836 A1
(43) Pub. Date: Mar. 13, 2014

(54) SYSTEMS AND METHODS FOR PROMOTING BONE GROWTH UTILIZING AN AUTOMATICALLY ADJUSTABLE, PROGRAMMABLE COMBINED MAGNETIC FIELD THERAPY

(71) Applicant: Zavation, LLC, Brandon, MS (US)

(72) Inventors: Jeffrey Johnson, Flowood, MS (US); John Lawrence Walker, Madison, MS (US); Joel Frank McCown, Philadelphia, MS (US)

(73) Assignee: Zavation, LLC, Brandon, MS (US)

(21) Appl. No.: 14/025,373

(22) Filed: Sep. 12, 2013

Related U.S. Application Data
(60) Provisional application No. 61/700,108, filed on Sep. 12, 2012.

(51) Int. Cl. A61N 1/32

(52) U.S. Cl. CPC A61N 1/326
USPC 600/13; 607/51

(57) ABSTRACT

The present invention relates to a system and method of applying a therapeutic electrical field to a bone region of a human being in order to promote healing and growth that that bone region. The present invention also provides a system that produces a combined magnetic field (CMF) that is precisely located and controlled across the entire treatment area requiring bone growth stimulation by utilizing bone dimensional input parameters to automatically calculate and drive the appropriate output therapy single through a single coil.

### RELATED APPLICATION

This application claims priority to U.S. Provisional Patent Application Ser. No. 61/700,108, filed Sep. 12, 2012, the entirety of which is hereby incorporated by reference for all purposes.

### TECHNICAL FIELD

The present invention relates to systems and methods for promoting bone growth using electrical energy.

### BACKGROUND

When utilized in Combined Magnetic Field (CMF) bone growth stimulators, the cyclotron resonance relationship detailed in U.S. Pat. No. 5,059,298 is effective when the entire region requiring bone growth is subjected to the CMF field containing the precise cyclotron resonance ratio for the selected ion(s). Dual coil CMF bone growth stimulators, like that described in U.S. Pat. No. 4,932,951, are adapted to produce this field, by placing the two coils opposite each other, similar to a Helmholtz configuration, such that the selected cyclotron resonance ratio is produced between the two coils across the entire region requiring treatment. However, single coil CMF bone growth stimulators, such as that described in U.S. Pat. No. 7,465,269, do not produce a CMF therapy in which the cyclotron ratio is constant across the region under treatment. This is because the magnitude of the magnetic field drops very quickly as the axial distance from the center of the coil increases. To illustrate this point, FIG. 3 depicts a representative single coil transducer configured and driven such that it produces a 76.6 Hz, 0.2 Gauss magnetic field at the center of the region requiring treatment, located 2.5 inches from the X-Y plane, along the Z-axis. Referring to FIG. 4, it can be seen that the magnetic field quickly drops as the distance along the Z-axis increases. The drop in magnetic field results in an increase in the cyclotron resonance ratio as seen in FIG. 5. This characteristic is problematic when considering that single coil devices are prescribed for use in patients of different sizes, weights, and sexes—parameters which greatly influences the distance from the center of the transducer coil to the region that requires bone growth therapy. This results in significant portions of bone under treatment being subjected to CMF therapies that do not match the cyclotron resonance ratio of the selected ion(s).

### SUMMARY

The present invention addresses the drawback of single coil CMF bone growth stimulators, by automatically calculating and adjusting the stimulation therapy parameters (such as, for example, static B-field magnitude, and AC frequency) based upon parameters that are preprogrammed into a control unit at a manufacturing facility or patient dimensional parameters that are measured and programmed into a control unit by a therapist such as a physician.

The present invention provide systems and methods of applying a therapeutic magnetic field to a bone region of a human being, such as a lumbar spinal region, in order to promote the healing and growth of lumbar vertebrae, or other relevant vertebrae, following spinal fusion surgery, such as lumbar spinal fusion surgery. More specifically, in an embodiment, the present invention provides a system that produces a CMF that is precisely located and controlled across the entire spinal region requiring bone growth stimulation by utilizing lumbar spine dimensional input parameters to automatically calculate and drive the appropriate output therapy signal through a single coil. This provides an improved method of applying a CMF bone growth therapy to the lumbar spinal region over other single coil devices, such as those described in U.S. Pat. Nos. 5,792,040 and 7,465,269, which are incorporated by reference herein. The CMF therapy described in these patents uses low frequency magnetic fields that are functionally dependent upon ion cyclotron resonance frequencies as described in U.S. Pat. No. 4,932,951, which is incorporated by reference herein.

### BRIEF DESCRIPTION OF THE DRAWINGS

FIG. 1 shows a front perspective view of an embodiment of a lumbar bone growth stimulator, according to the present invention.

FIG. 2 shows a front perspective cross-sectional view of an embodiment of the lumbar bone growth stimulator of FIG. 1, illustrating internal components of the stimulator according to the present invention.

FIG. 3 shows a front perspective view of a circular transducer coil lying in the X-Y plane, with the magnetic field directed along the Z-axis.

FIG. 4 is a graph that displays the magnetic field intensity of the circular transducer coil in FIG. 3, along the Z-axis as a function of the distance from the center of the circular transducer coil, which is configured to output a magnetic field of 0.2 Gauss at a distance of 2.5 inches. The dashed line indicates the ideal B-field created for CMF therapy when the AC field frequency is 76.6 Hertz.

FIG. 5 is a graph that displays the ratio of the cyclotron resonance frequency (AC frequency) to the magnitude of the static magnetic field (B-field) as a function of distance along the Z-axis for the circular transducer coil of FIG. 3. The dashed line indicates the ideal condition for CMF bone growth in which the AC frequency to B-field ratio is constant at 383 Hz/Gauss at all depths, with an AC frequency of 76.6 Hertz.

FIG. 6 show a right lateral view of the midsagittal plane of the lumbar region of a human being, with an embodiment of the lumbar transducer assembly 20 of the present invention located in the ideal position for bone growth stimulation of vertebrae L3. Line A is located in the plane in which the centers of the transducer assembly 20, transducer coil 21, and magnetic sensor 22 reside. Line B indicates the location of the human's posterior skin along Line E in the midsagittal plane. Line C indicates the location of the most posterior spinous processes of the vertebrae requiring treatment. Line D indicates the location of the most anterior vertebrae bodies requiring treatment. Line E is located centrally to the region requiring treatment (in this case, vertebrae L3). Lumbar belt 30 is not shown for clarity.

FIG. 7 is a graph that displays seven scatter plots of the B-field output (right-hand axis) at the center of the transducer coil 21 according to an embodiment of the present invention, along with the cyclotron resonance ratio curves associated with each B-field point. The dashed line indicates the ideal condition for CMF bone growth in which the AC frequency to B-field ratio is constant at 383 Hz/Gauss at all depths, with an AC frequency of 76.6 Hertz.

### DETAILED DESCRIPTION

The present invention provides systems and methods for promoting bone growth by automatically adjusting, and programming delivery of electrical energy, such as a combined magnetic field, to a patient in need of bone growth.

By “operative state,” is the state of the system when it is being used to treat a patient.

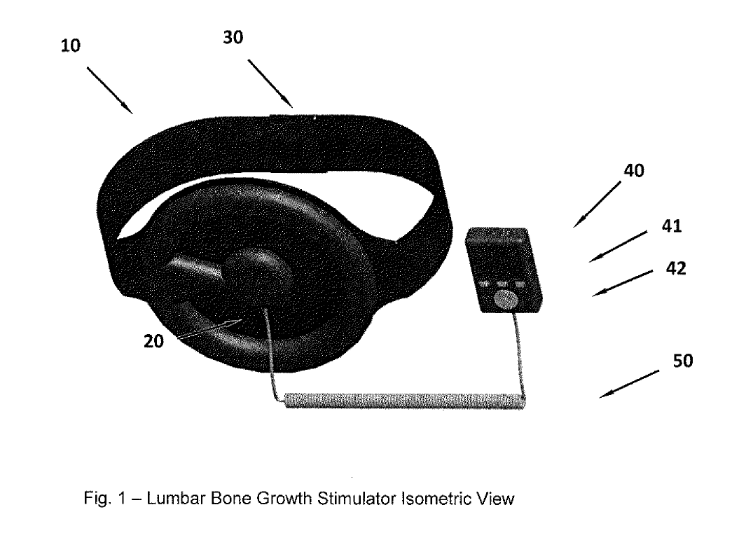

FIG. 1 shows a front perspective view of an exemplary lumbar bone growth stimulator device 10, which comprises lumbar transducer assembly 20, lumbar belt 30, control unit 40, and multi-conductor cable 50. Lumbar transducer assembly 20 delivers the combined magnetic field therapy to the lumbar spinal region, and is secured and held at the lumbar spinal region of a patient by lumbar belt 30, which is worn around the patient's midsection. This therapy can be initiated by the patient using control unit 40, which produces a coil drive signal and delivers it to lumbar transducer assembly 20 via multi-conductor cable 50. Control unit 40 can also be utilized by the physician in order to program the combined magnetic field output based upon the patient's lumbar dimensional parameters.

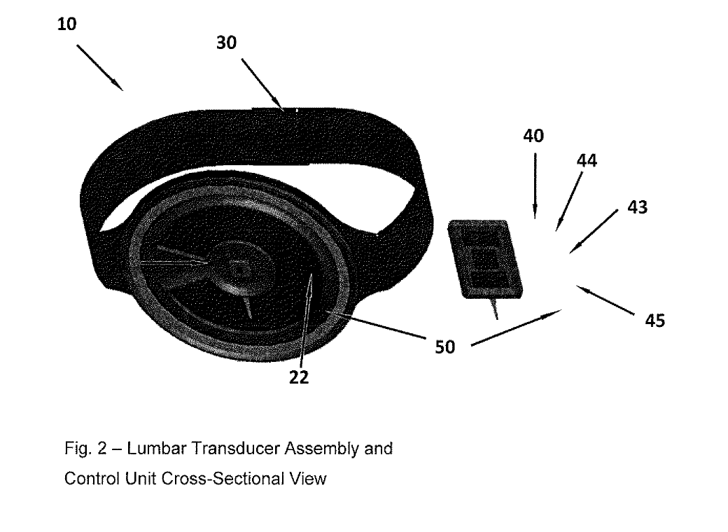

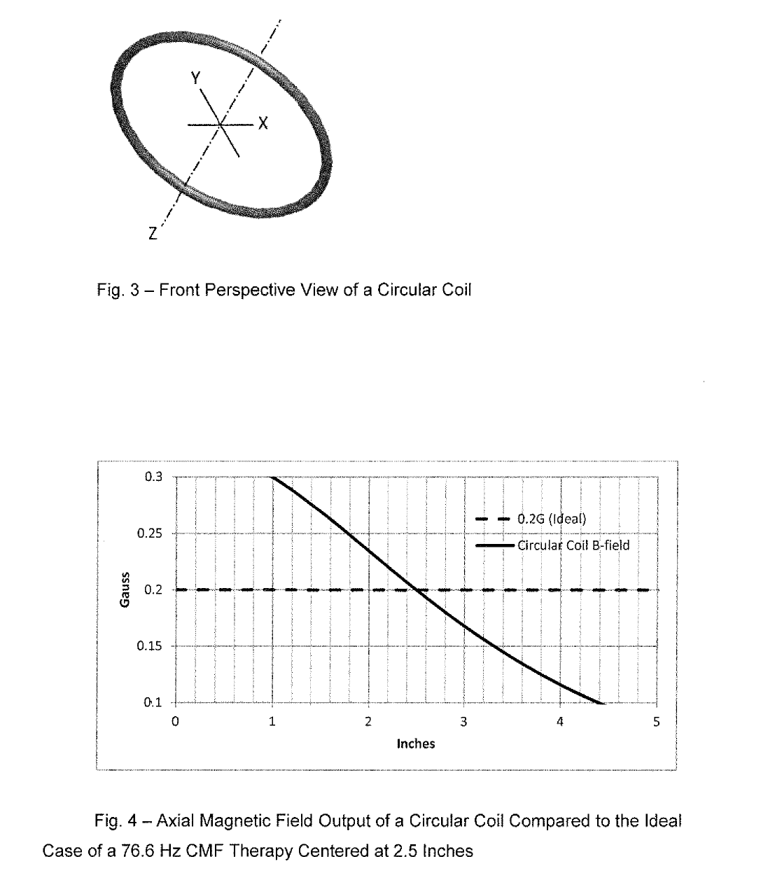

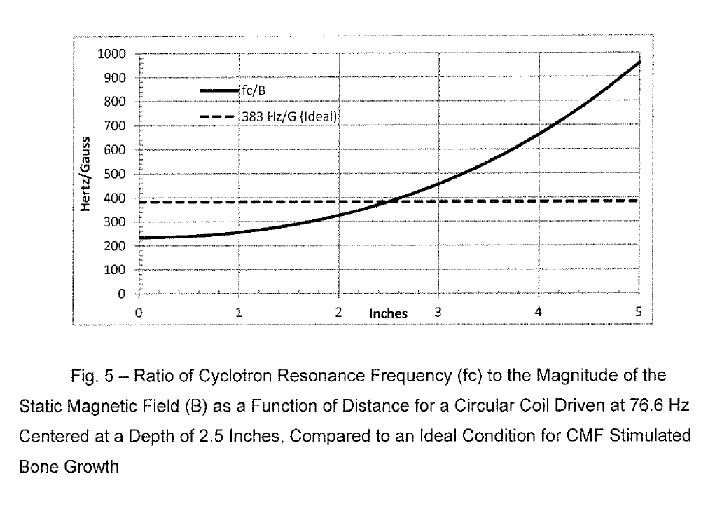

Referring to FIG. 2, lumbar transducer assembly 20 comprises an enclosed transducer coil 21 and magnetic sensor 22. Control unit 40 comprises drive circuit 44, processor 43, memory 45, display 41 and user-interface 42. Lumbar transducer assembly 20 delivers the CMF therapy to the lumbar spinal region through enclosed transducer coil 21, which comprises many turns of magnet wire. Transducer coil 21 is driven by drive circuit 44, whose output is automatically calculated and commanded by on-board processor 43, and delivered via multi-conductor cable 50. The CMF therapy produced by the transducer coil 21 is an automated, ramping, combined magnetic field which extends across the lumbar spinal region under treatment. Magnetic sensor 22 accurately measures the magnitude of the combined magnetic field that is output by transducer coil 21. The magnetic field measurements are reported back to control unit 40 via the multi-conductor cable 50. Processor 43 uses this magnetic feedback to accurately control the combined magnetic field magnitude and/or frequency such that the selected cyclotron resonance ratio is maintained. Control unit 40 also includes a user interface comprising a display 41, such as an LCD display, and pushbuttons 42. Display 41 can be used to display information such as, for example, system status, therapy status, and the programmed input data. Pushbuttons 42 are used by the physician to program the device 10, and by the patient and/or physician to activate and deactivate therapy sessions.

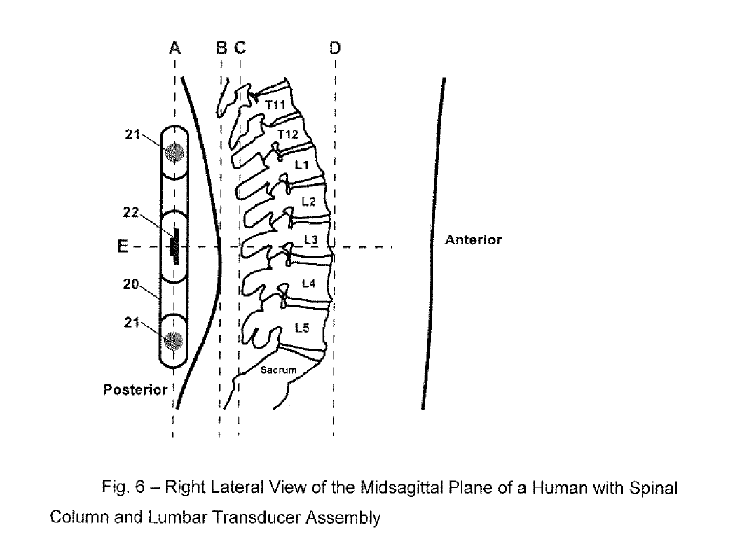

Prior to programming device 10 for operation, a physician can first makes measurements using, for example, manual and/or radiographic techniques. Referring to FIG. 6, a physician can determine the following default patient dimensional parameters:

Distance AB—the linear distance from the magnetic sensor 22 (line A) to the patient's posterior skin (line B) at the midsagittal plane, along line E, which is substantially vertically central to the magnetic sensor 22 and to the lumbar spinal region requiring treatment. By “substantially” is meant that the measurements need not be exact such as exactly central. Device 10 is fitted on the patient's lumbar back at substantially the center of the region requiring treatment in order to make this measurement.

Distance BC—the linear distance from the patient's posterior skin (line B) to the spinous process (line C) at the midsagittal plane, along line E, which is substantially vertically central to the magnetic sensor 22 and to the lumbar spinal region requiring treatment. This dimension is the minimum depth which requires bone growth therapy, and is determined using radiographic techniques such as, for example, X-rays, CT scans, and MRI's.

Distance BD—the linear distance from the patient's posterior skin (line B) to the anterior vertebral body (line D) at the midsagittal plane, along line E, which is substantially vertically central to the magnetic sensor 22 and to the lumbar spinal region requiring treatment. This dimension is the maximum depth which requires bone growth therapy, and is determined using radiographic techniques such as, for example, X-rays, CT scans, and MRI's.

While the above default dimensions have been selected to include the minimum and maximum lumbar spinal column depths at the region requiring treatment, it is understood that the physician has the flexibility to select minimum and maximum values case-by-case, based on any number of considerations. It is also understood that the linear distances could be measured from any point other than the magnetic sensor 22, such as substantially the center of the transducer coil 21, a surface of the lumbar transducer assembly 20, or any other reference point.

Once the dimensional parameters are determined, the physician can program these values into control unit 40 using user interface buttons 42. The dimensions are stored into the on-board memory 45 located in the control unit 40. Those skilled in the art understand that there exist various memory options which could be utilized to store this data, such as discrete memory integrated circuits, memory cards, or USB drives, among others. Also, the method by which the dimensions are entered by the physician could also be various, such as, for example, touchscreen displays, internal tactile switches, programming through an external computer, or other devices.

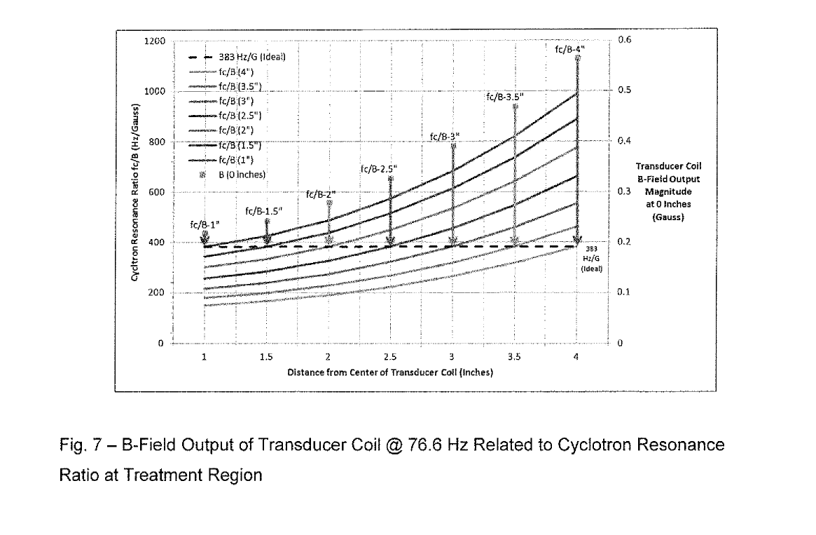

With the dimensional parameters stored in the memory 45 of control unit 40, processor 43 automatically calculates and drives the required range of CMF output parameters (static B-field magnitude, AC frequency, magnitude of AC field, etc.) such that the entire depth of lumbar vertebrae bone(s) requiring therapy is subjected to the selected cyclotron resonance ratio (in this case 383 Gauss per Hertz, assuming an AC frequency of 76.6 Hertz), in succession, from the minimum to the maximum programmed depths. Referring to FIG. 7, seven output curves are shown to illustrate the automatic movement of the selected cyclotron resonance ratio across the region of treatment. The seven scatter points represent seven B-fields that are produced at the center of transducer coil 21, and measured by magnetic sensor 22. The value of the B-fields are measured in Gauss, and displayed on the right-hand vertical axis. Each of these B-fields created at the center of transducer coil 21 results in a specific cyclotron resonance ratio curve as seen on the graph, and measured in Hertz per Gauss as displayed on the left-hand vertical axis. It can be seen, that as control unit 40 commands and increments the B-field from one scatter point to the next, the cyclotron resonance ratio curve moves such that the selected cyclotron resonance ratio (383 Hz/G) increases in depth. Processor 43 automatically calculates and adjusts the output B-field in time so that the entire region requiring treatment is subjected to the selected cyclotron resonance ratio at a preprogrammed rate. This rate could be constant or variable, and could repeat from minimum to maximum, maximum to minimum, or from minimum to maximum and back to minimum again. It should be noted that the B-field could be adjusted using various methods or algorithms other than a ramp function. It should also be noted that the number of increments need not be seven, but could be any number. As the number of increments increases, the CMF therapy becomes more precise in locating the selected cyclotron resonance ratio at all points across the region requiring treatment. In yet other embodiments, the selected cyclotron resonance ratio can be produced across the entire region requiring treatment by incrementing the AC frequency, or both the B-field magnitude and the AC frequency. The magnitude of the AC field can automatically adjust so that its peak-to-peak value is 0.4 Gauss at a depth of 6 inches.

The cycling of CMF therapy described herein, differs from existing combined magnetic field bone growth stimulators in that the selected cyclotron resonance ratio moves across the region of treatment (such as 0 to 6 inches from the transducer housing). This allows the treatment to cover the entire lumbar spinal column depth of most patients at a pre-determined cycle velocity. The result is that each point along the treatment span will be subjected to the precise cyclotron resonance ratio for only a brief portion of the therapy session—roughly 200 milliseconds for each 20 mil section per cycle in the preferred embodiment. CMF bone growth technology previously known in the art, works by subjecting a specific region of treatment, such as the transverse processes or the area between two coils in the case of dual coil stimulator to the precise cyclotron resonance ratio continuously at a fixed depth for the entire therapy session. However, the present invention relates to bone growth achieved using a cycling combined magnetic field, even though each section of the region of treatment is not subjected to a continuous cyclotron resonance ratio.

It is understood that the dimensional parameters could be preprogrammed at the factory such that minimum and maximum treatment distances would accommodate patients of all shapes, sizes, and sexes. This embodiment would eliminate the need for the physician to make manual and radiographic measurements, and program this data into the device 10. A similarly effective magnetic field output could be produced by programming a single depth into the control unit 40, which would then automatically calculate and adjust the stimulation parameters such that the entire region requiring treatment is subjected to the selected cyclotron resonance ratio.

Although the present invention details a CMF lumbar device, the methods and techniques provided herein could be utilized for any bone growth stimulation technology (pulsed electromagnetic field, capacitive coupling, direct current, ultrasound, etc.), or any bone growth stimulation application (cervical, tibia, femur, scaphoid, humerus, ulna, fibula, etc.). Further, the techniques provided herein can be utilized for multi-coil bone growth stimulators.

Finally, the cyclotron resonance ratio of 383 Gauss per Hertz described herein is derived from the 3^{rd} and 5^{th} harmonics of the cations Mg²⁺ and Ca²⁺ respectively, as detailed in “The Charge-to-Mass ICR Signature in Weak ELF Bioelectromagnetic Effects” by Abraham R., which is also incorporated by reference herein. Also, any number of cyclotron resonance relationships related to bone growth can be utilized, as detailed in the above reference and in U.S. Pat. No. 4,932,951, which is incorporated herein

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A bone growth stimulation system therapy for stimulating bone growth in treatment region of a patient comprising:
a transducer assembly comprising a transducer coil; and
a control unit programmed to produce an output signal to the transducer coil, wherein the output signal produced by the transducer coil is cycled across various depths of the entire treatment region in an operative state.

2. The system of claim 1, wherein the electrical field is a combined magnetic field.

3. The system of claim 1, wherein the control unit is pre-programmed at a factory with minimum and maximum treatment distances to accommodate patients of different shapes, sizes, and sexes.

4. The system of claim 3, wherein the minimum distance is 0 inches and the maximum distance is six inches.

5. The system of claim 1, wherein the control unit is manually programmed by a physician with measurements obtained from the patient based on the patient's lumbar dimensional parameters.

6. The system of claim 1, further comprising a lumber belt attached to the lumber transducer assembly.

7. The system of claim 1, wherein the magnetic sensor is configured to measure the magnitude of the combined magnetic field that is output by the transducer coil in an operative state.

8. The system of claim 7, wherein the magnetic field measurements are reported back to the control unit via the cable in an operative state.

9. The system of claim 7, wherein the processor uses this magnetic feedback to control the combined magnetic field magnitude and/or frequency such that the selected cyclotron resonance ratio is maintained in an operative state.

10. The system of claim 1, wherein a physician determines default patient dimensional parameters of the system.

11. The system of claim 10, wherein the default patient parameters include the linear distance from a magnetic sensor to the patient's posterior skin in the midsagittal plane;
the linear distance from the patient's posterior skin to the spinous process at the midsagittal plane; and the linear distance from the patient's posterior skin to an anterior vertebral body at the midsagittal plane.

12. The system of claim 1, wherein with dimensional parameters of the patient stored in the memory of the control unit, the processor automatically calculates and drives the required range of CMF output parameters such that the entire depth of lumbar vertebrae bone(s) requiring therapy is subjected to the selected cyclotron resonance ratio, in succession, from the minimum to the maximum programmed depths in operative state.

13. The method of claim 12, wherein the cyclotron resonance ratio is 383 Gauss per Hertz assuming an AC frequency of 76.6 Hertz.

14. The system of claim 10, wherein the CMF output parameters are static B-field magnitude, AC frequency, and the magnitude of the AC field.

15. The system of claim 12, wherein the therapy is produced with a constant frequency to B-ratio and cycling the therapy across all depths in the region of treatment in an operative state.

16. The system of claim 12, wherein bone growth is enhanced by cycling the selected frequency to B-field ratio across the treatment span at a specific cycle velocity.

17. The system of claim 16, wherein the selected therapy moved from 0 to six inches once every 60 seconds for a total of 12 times over a 1800 treatment session yield a 0.1 inches/second cycle velocity.
